(19) United States
(12) Patent Application Publication
(10) Pub. No.: US 2023/0248787 A1
(43) Pub. Date: Aug. 10, 2023

(54) PROBIOTIC STRAIN SELECTED BY TARGETED IN VIVO ENRICHMENT TO AID WITH HEALTHY LACTOSE DIGESTION

(71) Applicant: Synbiotic Health, Inc., Lincoln, NE (US)

(72) Inventors: Jens Walter, Cork (IE); Zachery Lewis, American Fork, UT (US); Thomas Auchtung, Lincoln, NE (US)

(21) Appl. No.: 18/163,733

(22) Filed: Feb. 2, 2023

Related U.S. Application Data
(60) Provisional application No. 63/306,547, filed on Feb. 4, 2022.

(51) Int. Cl. A61K 35/745; C12N 1/20; C12N 9/38; A23L 33/135; A61P 1/14

(52) U.S. Cl. CPC A61K 35/745; C12N 1/20; C12N 9/2471; A23L 33/135; A61P 1/14; C12Y 302/01108

(57) ABSTRACT

Methods and compositions for a probiotic strain selected by targeted in vivo (in the human gut) enrichment to aid with healthy lactose digestion are provided. The probiotic strain selected may beneficially include Bifidobacterium adolescentis strain IVS-1 for reducing and/or preventing symptoms of lactose intolerance in a subject in need thereof.

### CROSS-REFERENCE TO RELATED APPLICATIONS

This application claims the benefit of U.S. Provisional Application Ser. No. 63/306,547, filed on Feb. 4, 2022, and entitled “Probiotic Strain Selected by Targeted In Vivo (In the Human Gut) Enrichment to Aid with Healthy Lactose Digestion,” the entire contents of which are incorporated herein by reference.

### SEQUENCE LISTING XML

The instant application contains a sequence listing, which has been submitted in XML file format by electronic submission and is hereby incorporated by reference in its entirety. The XML file, created on Feb. 2, 2023, is named 93259.2.USU1_Sequence Listing.xml and is 3305 bytes in size.

### TECHNICAL FIELD

The disclosure relates to compositions and methods of supporting lactose digestion, including the incorporation of a probiotic, such as, therapeutically effective amounts of an isolated Bifidobacterium adolescentis strain IVS-1.

### BACKGROUND

Dairy foods are an important source of protein, riboflavin, and calcium, particularly in the United States, Europe, Asia, Africa, and many other areas of the world. However, many individuals suffer from lactose intolerance. This condition results from the absence or insufficient production of the enzyme lactase, also known as beta-galactosidase. Acquired lactase deficiency is the most common disorder of complex carbohydrate absorption throughout the world, affecting up to 75% percent of the world's population. In the United States, 15% of Caucasians, over 50% of Hispanics, and over 80% of African Americans suffer from lactose intolerance. The disorder is characterized by gastrointestinal symptoms of excessive gas production, abdominal pain, cramps, bloating, and diarrhea after excessive consumption of lactose-containing foods such as dairy and dairy-based products.

In infancy, mammals have a high level of lactase activity in the lining of the upper intestinal tract, because they depend on lactose as the primary carbohydrate in their diet. However, in humans, lactase expression in the upper intestinal tract is diminished by about 90% between ages two and five. This condition is called primary lactase deficiency. Many Northern Europeans, some Western Europeans, Mediterraneans, and their descendants carry a mutation that prevents this natural decrease in lactase production. These individuals are able to consume milk and other dairy-based products as adults without problem. However, large portions of the world population, such as Southern Europeans, East Asians, and Sub-Saharan Africans have primary lactase deficiency.

Alternatively, secondary lactase deficiency results from injury or disease of the small intestine. For example, celiac disease, inflammatory bowel syndrome (IBS) and Crohn's disease are often accompanied by lactase deficiency. These diseases occur in all ethnic groups.

Lactose is a disaccharide of glucose and galactose linked by a beta-D-glycosidic bond. The disaccharide is digested into its individual sugars by the lactase (beta-D-galactosidase) produced in the small intestine by the cells of the intestinal brush border. Because glucose and galactose are absorbed in the small intestine, when lactase is absent from the small intestine, undigested lactose reaches the large intestine. There, the resident bacteria metabolize lactose through fermentation, possibly generating gas within the large intestine depending on the specific bacterial species. The gas is responsible for symptoms such as pain, pressure, cramps and flatulence. In addition, the undigested lactose increases osmotic pressure in the intestine, causing increased excretion of water and diarrhea.

One way to supplement lactase involves ingesting live or killed lactase-producing bacteria. For example, it is known that persons with mild lactose intolerance are able to tolerate yogurt but not milk, although both products contain the same amount of lactose. This is due to the fact that bacteria added to yogurt, such as Streptococcus, Lactobacillus and Bifidobacterium species, express functional lactase both during the fermentation as well as during digestion in the intestinal tract. Thus, to improve the ability to digest lactose, one may consume yogurt products containing live and active cultures of these bacteria.

Unfortunately, physicians report limited success with treating lactose intolerance with many traditional probiotic bacterial cultures. The review of the literature conducted by family practitioners at the University of Pittsburg (“Do probiotics reduce adult lactose intolerance?” J. Fam. Pract., 2005; v. 54, No. 7, p. 613-620) concluded that overall, the strategy was ineffective. The authors suggest that this is due to variation in bacterial viability and ability to produce lactase between the different dairy products and supplements. According to the study, with a few exceptions, most of these products do not provide sufficient lactase activity once in the human body to alleviate the symptoms of lactose intolerance. Other attempts to solve lactose intolerance with probiotics have used non-native strains that cannot 1) survive GI transit, 2) colonize the gut, or 3) compete for lactose in the ecological context of the gut. Probiotic strains that don't survive the transit may mitigate symptoms via release of active lactase into the lumen of the gut when they lyse—but are unlikely to provide long term relief. Similarly, probiotics that can't colonize will also eventually wash through the gut without providing longer-term alleviation of symptoms. Strains that aren't competitive for the lactose in situ may still allow undesirable species access to the substrate, potentially leading to undesirable gas production, or not metabolize the substrate quickly enough to prevent osmotic effects from causing watery stool. Attempts to demonstrate a solution to lactose intolerance via supplementation with a lactose-like prebiotic galactooligosaccharide (GOS) were not ultimately successful, such as the Phase 3 Trial data presented by Ritter Pharmaceuticals (https://www.globenewswire.com/news-release/2019/09/12/1915140/0/en/Ritter-Pharmaceuticals-Reports-Top-Line-Results-from-its-Liberatus-Phase-3-Trial-of-RP-G28-for-Lactose-Intolerance.html) likely due to some combination of 1) individual variation in the microbiome of subjects and perhaps a lack of enrichable and competitive lactose-degrading strains, and 2) competition of the GOS with dietary lactose for metabolism by resident lactose-degrading bacteria.

The symptoms of lactose intolerance were not relieved when treatment with a very common probiotic species, Lactobacillus acidophilus was used. Among common yogurt additive bacteria, L. acidophilus has one of the highest natural levels of lactase and the ability to adhere to the intestinal wall. Nevertheless, a Tufts University study “A randomized trial of Lactobacillus acidophilus BG2F04 to treat lactose intolerance”, Am. J. Clin. Nutr. 1999, 69:140-146, concluded that even this bacterium was ineffective against the symptoms of lactose intolerance.

There remains a need in the art for improved methods and compositions of alleviating or preventing symptoms of lactose intolerance.

### SUMMARY

Provided herein are methods and compositions for a probiotic strain selected by targeted in vivo (in the human gut) enrichment to aid with healthy lactose digestion.

In Example 1, a method of treating lactose intolerance comprises administering to a subject in need thereof a therapeutically effective amount of an isolated Bifidobacterium adolescentis strain IVS-1.

Example 2 relates to the method according to Example 1, wherein the strain is administered in an amount of at least 10⁶ CFU per day.

Example 3 relates to the method according to Example 1 or 2, wherein the strain is administered daily for one week or longer.

Example 4 relates to the method according to any one of Examples 1 to 3, wherein the method improves lactose degradation and/or decreases lactose intolerance symptoms in a subject in need thereof.

Example 5 relates to the method according to any one of Examples 1 to 4, wherein the Bifidobacterium adolescentis strain IVS-1 is pre-treated with lactose to increase initial lactase activity of the Bifidobacterium adolescentis strain IVS-1.

Example 6 relates to the method according to any one of Examples 1 to 5, wherein the method provides lactase to a subject in need thereof via administration of both live and inactive cultures of Bifidobacterium adolescentis strain IVS-1 and the actual and former cellular contents thereof.

Example 7 relates to the method according to any one of Examples 1 to 6, wherein the method reduces gas production in the intestines.

Example 8 relates to the method according to any one of Examples 1 to 7, wherein the method balances osmotic pressures in the intestines.

Example 9 relates to the method according to any one of Examples 1 to 8, wherein the method alters the intestinal microbiome to benefit the health and wellbeing of a subject in need thereof.

In Example 10, a food composition to treat lactose intolerance comprises Bifidobacterium adolescentis strain IVS-1, wherein the inoculum of Bifidobacterium adolescentis strain IVS-1 demonstrates lactase activity.

Example 11 relates to the composition according to Example 10, wherein the food composition is selected from the group consisting of milk, curd, milk based fermented products, acidified milk, yoghurt, desserts, snack foods, chocolates, candy, lozenges, frozen yoghurt, milk powder, milk based powders, milk concentrate, cheese, cheese spreads, dressings, beverages, ice-creams, bars, additive powders, encapsulated and tabled supplements, fermented or non-fermented cereal based products, infant formulae, tablets, liquid bacterial suspensions, dried oral supplement, and wet oral supplement.

Example 12 relates to the composition of according to Example 10 or 11, further comprising a pharmaceutically acceptable carrier.

Example 13 relates to the composition according to any one of Examples 10 to 12, further comprising a prebiotic selected from the group consisting of: galactooligosaccharides (GOS), fructooligosaccharides (FOS), and inulin, mannan-oligosaccharides (MOS), arabinoxylans, or a composition obtained from the bacterial cells, metabolites, enzyme activities, and polysaccharides of Bifidobacterium adolescentis strain IVS-1.

Example 14 relates to the composition according to any one of Examples 10 to 13, wherein Bifidobacterium adolescentis strain IVS-1 is selected from a wild type strain, a derived strain, and/or a mutant strain.

Example 15 relates to the composition according to any one of Examples 10 to 14, wherein Bifidobacterium adolescentis strain IVS-1 is at a concentration at about 10⁴ to about 10¹⁴ CFU.

In Example 16, a method of treating lactose intolerance comprises administering to a subject in need thereof a therapeutically effective amount of a Bifidobacterium adolescentis strain IVS-1; and increasing the lactase activity by Bifidobacterium adolescentis strain IVS-1 in the gastrointestinal tract after at least 1 day of administration.

Example 17 relates to the method according to Example 16, wherein the method further comprising increasing the beta-galactosidase activity levels in the gastrointestinal tract via administration of Bifidobacterium adolescentis strain IVS-1.

Example 18 relates to the method according to Example 16 or 17, wherein the method further comprises decreasing the lactose levels in the gastrointestinal tract via administration of Bifidobacterium adolescentis strain IVS-1.

Example 19 relates to the method according to any one of Examples 16 to 19, wherein the method further comprises delivering an effective amount of GOS with the Bifidobacterium adolescentis strain IVS-1.

Example 20 relates to the method according to any one of Examples 16 to 19, wherein the method further comprises decreasing hydrogen production in the gastrointestinal tract and reducing symptoms related to lactose intolerance in the subject.

While multiple embodiments are disclosed, still other embodiments of the disclosure will become apparent to those skilled in the art from the following detailed description, which shows and describes illustrative embodiments of the disclosure. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

In the accompanying figures, like elements are identified by like reference numerals among the several preferred embodiments of the present disclosure.

FIG. 1 is a graph showing the growth of Bifidobacterium adolescentis strain IVS-1 in comparison to a number of other probiotic strains when grown in a gut-like media containing lactose.

FIG. 2 is a graph showing the Bifidobacterium adolescentis strain IVS-1 providing greater reduction of gas production from lactose-spiked fecal samples in comparison to other probiotic strains.

FIG. 3 is a graph showing the expected results with treatment with Bifidobacterium adolescentis strain IVS-1 lowering hydrogen production and symptom scores.

FIG. 4 is a flowchart showing the clinical trial pathway for the visits, dietary washout, baseline measures and lactose challenge, treatment with Bifidobacterium adolescentis strain IVS-1 or placebo, and post treatment measures and lactose challenge for Example 4.

FIG. 5 is a graph showing the expected results of relative lactase activity of various probiotics and various growth conditions.

### DETAILED DESCRIPTION

The foregoing and other features and advantages of the disclosure are apparent from the following detailed description of exemplary embodiments, read in conjunction with the accompanying drawings. The detailed description and drawings are merely illustrative of the disclosure rather than limiting, the scope of the disclosure being defined by the appended claims and equivalents thereof.

Embodiments of the disclosure will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the disclosure. Furthermore, embodiments of the disclosure may include several novel features, no single one of which is solely responsible for its desirable attributes or which is essential to practicing the disclosure described herein.

The use of the terms “a” and “an” and “the” and similar referents in the context of describing the disclosure are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of about one log from the stated numerical value. For example, the term “about” may be construed to indicate a deviation of between 0.1 to 10 times the stated value. The use of any and all examples, or exemplary language (“e.g.” or “such as”) provided herein, is intended merely to better illuminate the disclosure and does not pose a limitation on the scope of the disclosure unless otherwise claimed. No language in the specification should be construed as indicating any nonclaimed element as essential to the practice of the disclosure.

References to “one embodiment,” “an embodiment,” “example embodiment,” “various embodiments,” etc., may indicate that the embodiment(s) of the disclosure so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used herein, “isolated” refers to the obtaining of a population of microbial cells in which at least about 80% (e.g., about 85%, 90%, 95%, 99% or 100%) of the cells are of a particular strain, such as the Bifidobacterium adolescentis strain IVS-1 described herein.

The expression “probiotics” is referred to herein as a composition which comprises probiotic microorganisms. Probiotic bacteria are defined as live bacteria, which when administered in adequate amounts confer a health benefit on the host. Probiotic microorganisms have been defined as “Live microorganisms which when administered in adequate amounts confer a health benefit on the host” (FAO/WHO 2002).

The expression “prebiotic” is referred to a composition or a component of a composition which is selectively utilized by host microorganisms conferring a health benefit”. Prebiotics are generally non-viable food components that are specifically fermented in the colon by bacteria thought to be of positive value, e.g., bifidobacteria, lactobacilli, and other short-chain fatty acid producing microorganisms. Prebiotics are also known as an ingredient that allows specific changes, both in the composition and/or activity in the gastrointestinal microbiota that confers benefits upon host well-being and health. The combined administration of a probiotic strain with one or more prebiotic compounds, when designed optimally, may enhance the growth of the administered probiotic in vivo resulting in additional or more pronounced health benefits, and is termed a synbiotic. A synbiotic is formally defined as “a mixture, comprising live microorganisms and substrate(s) selectively utilized by host microorganisms, that confers a health benefit on the host”. Well characterized prebiotics include, for example, galactooligosaccharide (GOS), fructooligosaccharide (FOS), and inulin. GOS and FOS refer to a group of oligomeric, non-digestible carbohydrates that are often produced from monomers using glycosidases to catalyze transgalactosylation reactions. These carbohydrates are often recalcitrant to digestion by host-secreted enzymes in the small intestine, such that they reach the colon intact and are available to the colonic microbiota. It would be understood by those skilled in the art that other compounds that fall within the definition of a prebiotic also can be used in the methods described herein.

As used herein, a “subject” can refer to a human or a non-human. Representative non-human subjects include, without limitation, livestock (e.g., swine, cow, horse, goat, and sheep), poultry (e.g., fowls such as chicken and turkey), and companion animals (e.g., pets such as dogs and cats).

In a further embodiment, the composition further comprises a pharmaceutically acceptable carrier. As used herein, the term “pharmaceutically acceptable carrier” means one or more solid or liquid filler diluents or encapsulating substances which are suitable for administration to a human or an animal and which is/are compatible with the active probiotic organisms. The term “compatible” relates to components of the pharmaceutical composition which are capable of being comingled with the Bifidobacterium adolescentis strain IVS-1 further described herein, or a mutant strain thereof in a manner enabling no interaction that would substantially reduce the probiotic efficacy of the organisms selected for the present disclosure under ordinary use conditions. Pharmaceutically acceptable carriers must be of a sufficiently high purity and a sufficiently low toxicity to render them suitable for administration to humans and animals being treated.

A bacterial “strain” as used herein refers to a bacterium which remains genetically unchanged when grown or multiplied. The multiplicity of identical bacteria is included. “Wild type strain” refers to the non-mutated form of a bacterium, as found in nature. In the present context, the term “derived strain” should be understood as a strain derived from a mother strain by means of e.g., genetic engineering, normal laboratory and commercial production culturing, radiation and/or chemical treatment, and/or selection, adaptation, screening, etc. In specific embodiments the derived strain is a functionally equivalent mutant, e.g., a mutant that has substantially the same, or improved, properties (e.g., regarding probiotic properties) as the mother strain. Such a derived strain is a part of the present disclosure. The term “derived strain” includes a strain obtained by subjecting a strain of the present disclosure to any conventionally used mutagenization treatment including treatment with a chemical mutagen such as ethane methane sulphonate (EMS) or N-methyl-N′-nitro-N- nitroguanidine (NTG), UV light, or to a spontaneously occurring mutant.

A “mutant bacterium” or a “mutant strain” refers to a natural (spontaneous, naturally occurring) mutant bacterium or an induced mutant bacterium comprising one or more mutations in its genome (DNA) which are absent in the wild type DNA. An “induced mutant” is a bacterium where the mutation was induced by human treatment, such as treatment with any conventionally used mutagenization treatment including treatment with chemical mutagens, such as a chemical mutagen comprising (i) a mutagen that associates with or become incorporated into DNA such as a base analogue, e.g., 2-aminopurine or an interchelating agent such as ICR-191, or (ii) a mutagen that reacts with the DNA including alkylating agents such as nitrosoguanidine or hydroxylamine, or ethane methyl sulphonate (EMS) or N-methyl-N′-nitro-N-nitroguanidine (NTG), UV- or gamma radiation etc. In contrast, a “spontaneous mutant” or “naturally occurring mutant” has not been mutagenized by man. A derived strain, such as a mutant, may have been subjected to several mutagenization treatments (a single treatment should be understood one mutagenization step followed by a screening/selection step), but typically no more than 20, no more than 10, or no more than 5, treatments are carried out. In specific embodiments of derived strains, such as mutants, less than 1%, less than 0.1%, less than 0.01%, less than 0.001% or even less than 0.0001% of the nucleotides in the bacterial genome have been changed (such as by replacement, insertion, deletion, or a combination thereof) compared to the mother strain. Mutant bacteria as described above are non-GMO, i.e., not modified by recombinant DNA technology. As an alternative to the above preferred method of providing the mutant by random mutagenesis, it is also possible to provide such a mutant by site-directed mutagenesis, e.g., by using appropriately designed PCR techniques or by using a transposable element which is integrable in bacterial replicons. When the mutant is provided as a spontaneously occurring mutant the above wild-type strain is subjected to the selection step without any preceding mutagenization treatment.

Compositions

The present disclosure provides for compositions and methods of utilizing probiotics for addressing negative symptoms associated with gas-producing and undesirable species in the gastrointestinal tract of a subject. In embodiments, the probiotic comprises Bifidobacterium adolescentis. In preferred embodiments, the probiotic comprises Bifidobacterium adolescentis strain IVS-1. Bifidobacterium adolescentis strain IVS-1 is an ecologically-adapted probiotic strain with the genetic capacity to be competitive for lactose and lactose-like carbohydrates in the human gut. Bifidobacterium adolescentis strain IVS-1 was identified by the process of in vivo selection (IVS) after being enriched in the human gut through consumption of the probiotic GOS (which is chemically similar to lactose) by an individual who naturally hosted the strain. Therefore, the Bifidobacterium adolescentis strain IVS-1 disclosed herein are provided at a concentration level much higher than would be found naturally within an individual subject. When administered in levels sufficient for colonization, the strain can outcompete gas-producing and other undesirable species in the human gut for the target carbohydrates. In aspects, Bifidobacterium adolescentis strain IVS-1 compensates for the lack of endogenous human lactase activity in the gut environment; thus reducing and/or eliminating the gastrointestinal symptoms of lactose intolerance. The reduction and/or elimination of gastrointestinal symptoms of lactose intolerance allows for people to eat more varied and dairy-rich diets.

The disclosure provides for the use of a Bifidobacterium adolescentis strain designated IVS-1, in which an isolate is deposited with the American Type Culture Collection under Accession No. PTA-120614. In some aspects, the present disclosure may comprise substantially similar strains with more than 95% similarity to the isolate deposited with the American Type Culture Collection under Accession No. PTA-120614. The use of a Bifidobacterium adolescentis strain IVS-1, or substantially similar strains thereof, can reduce and/or eliminate the undesirable intestinal effects of lactose on individuals with low or absent human lactase expression in the gut. A thorough description of the Bifidobacterium adolescentis strain IVS-1 is further available in U.S. Pat. No. 9,125,935, which is herein incorporated by reference.

Preferably, the present composition contains a Bifidobacterium adolescentis strain IVS-1 which has at least 95% identity with the 16S rRNA gene sequence when compared to the type strain identified in SEQ ID NO:1 below. In embodiments, the present composition contains Bifidobacterium adolescentis strain IVS-1 which has at least 97% identity, at least 98% identity, at least 98.5% identity, at least 99% identity, at least 99.5% identity, or at least 99.9% identity with the 16S rRNA gene sequence when compared to the type strain identified in SEQ ID NO:1 below, which is further provided within a sequence listing in XML format, incorporated herein by reference.

Sequence of 16S rRNA gene from microbial strain
Bifidobacterium adolescentis strain IVS-1
(SEQ ID NO: 1)
TGCAGTCGAA CGGGATCCCA GGAGCTTGCT CCTGGGTGAG
AGTGGCGAAC GGGTGAGTAA TGCGTGACCG ACCTGCCCCA
TACACCGGAA TAGCTCCTGG AAACGGGTGG TAATGCCGGA
TGCTCCAGTT GACCGCATGG TCCTCTGGGA AAGCTTTTGC
GGTATGGGAT GGGGTCGCGT CCTATCAGCT TGATGGCGGG
GTAACGGCCC ACCATGGCTT CGACGGGTAG CCGGCCTGAG
AGGGCGACCG GCCACATTGG GACTGAGATA CGGCCCAGAC
TCCTACGGGA GGCAGCAGTG GGGAATATTG CACAATGGGC
GCAAGCCTGA TGCAGCGACG CCGCGTGCGG GATGACGGCC
TTCGGGTTGT AAACCGCTCT TGACTGGGAG CAAGCCCTTC
GGGGTGAGTG TACCTTTCGA ATAAGCACCG GCTAACTACG
TGCCAGCAGC CGCGGTAATA CGTAGGGTGC AAGCGTTATC
CGGAATTATT GGGCGTAAAG GGCTCGTAGG CGGTTCGTCG
CGTCCGGTGT GAAAGTCCAT CGCTTAACGG TGGATCCGCG
CCGGGTACGG GCGGGCTTGA GTGCGGTAGG GGAGACTGGA
ATTCCCGGTG TAACGGTGGA ATGTGTAGAT ATCGGGAAGA
ACACCAATGG CGAAGGCAGG TCTCTGGGCC GTCACTGACG
CTGAGGAGCG AAAGCGTGGG GAGCGAACAG GATTAGATAC
CCTGGTAGTC CACGCCGTAA ACGGTGGATG CTGGATGTGG
GGACCATTCC ACGGTCTCCG TGTCGGAGCC AACGCGTTAA
GCATCCCGCC TGGGGAGTAC GGCCGCAAGG CTAAAACTCA
AAGAAATTGA CGGGGGCCCG CACAAGCGGC GGAGCATGCG
GATTAATTCG ATGCAACGCG AAGAACCTTA CCTGGGCTTG
ACATGTTCCC GACAGCCGTA GAGATACGGT CTCCCTTCGG
GGCGGGTTCA CAGGTGGTGC ATGGTCGTCG TCAGCTCGTG
TCGTGAGATG TTGGGTTAAG TCCCGCAACG AGCGCAACCC
TCGCCCTGTG TTGCCAGCAC GTCGTGGTGG GAACTCACGG
GGGACCGCCG GGGTCAACTC GGAGGAAGGT GGGGATGACG
TCAGATCATC ATGCCCCTTA CGTCCAGGGC TTCACGCATG
CTACAATGGC CGGTACAACG GGATGCGACA CTGTGAGGTG
GAGCGGATCC CTTAAAACCG GTCTCAGTTC GGATTGGAGT
CTGCAACCCG ACTCCATGAA GGCGGAGTCG CTAGTAATCG
CGGATCAG

In some aspects, measured by 16S rRNA gene sequencing. B. adolescentis (such as strain IVS-1) was identified as a taxa of interest that appeared to increase upon lactose feeding, as shown in Table 1. Operational taxonomic units (OTUs) constructs “mathematically” defined taxa, which is widely accepted and applied to describe bacterial communities using amplicon sequencing of 16S rRNA gene.

TABLE 1
Proportions of bacterial taxa (with >0.1% in at least one of the treatments)
significantly influenced by dietary treatments within treatment groups
(FDR-adjusted P value < 0.1). Significant values are set in italics

| | | | | | Q value |
|---|---|---|---|---|---|
| | | | | P | (FDR |
| Treatment | Taxonomic Group | Baseline | Treatment | value | adjusted) |
| Lactose | OTUs^{a} | | | | |
| | OTU_1 (B. adolescentis) | 3.793 ± 7.47 | 6.645 ± 8.83 | 0.1221 | 0.1587 |
| | OTU_167 (B. animalis | 0.000 ± 0.00 | 0.203 ± 0.85 | 0.4504 | 0.5489 |
| | subsp. lactis) | | | | |
| | Phylum | | | | |
| | Actinobacteria | 10.471 ± 6.75 | 14.502 ± 6.89 | 0.0529 | 0.0794 |
| | Genus | | | | |
| | Bifidobacterium | 7.895 ± 8.10 | 12.008 ± 8.02 | 0.0917 | 0.1233 |
| | OTUs^{a} | | | | |
| | OTU_1 (B. adolescentis) | 1.241 ± 1.93 | 2.557 ± 4.11 | 0.6562 | 0.7755 |
| | OTU_167 (B. animalis | 0.000 ± 0.00 | 0.037 ± 0.14 | 0.7910 | 0.8814 |
| | subsp. lactis) | | | | |
| | OTU_315 (Coprobacillus) | 0.100 ± 0.09 | 0.062 ± 0.06 | 0.0023 | 0.0179 |
| | OTU_43 (Ruminococcus2) | 0.562 ± 0.75 | 0.272 ± 0.63 | 0.0051 | 0.0284 |
| | OTU_180 (Bacteroides) | 0.041 ± 0.17 | 0.127 ± 0.40 | 0.0183 | 0.0476 |

^{a}If the strain could not be assigned to a type strain (<97% homology), RDP Classifier was used to determine the most likely genus (80% cutoff)
*Significant difference between the two treatment groups for OTU_1.

In one embodiment, the present composition comprises a mutant strain of any of the B. adolescentis strains with accession number PTA-120614, which can be obtained by subjecting the strain to mutagenization treatment as described to obtain mutant strains. In some aspects, mutant strains are selected for having desired properties such as the activity of specific enzymes, tolerance to bile and acid, increased fermentation yield, and superior shelf stability. Alternatively, a selection is performed for spontaneously occurring mutants. Also alternatively, embodiments comprising derivations of the strain resulting from the natural genetic drift that occurs over passaging of the strain during normal routine laboratory and commercial production activities may be utilized in the composition or methods of the present disclosure.

Beneficially, Bifidobacterium adolescentis strain IVS-1 colonizes the human gut or gastrointestinal tract at least 10 times better than a non-adapted strain of the same genus. In some aspects, Bifidobacterium adolescentis strain IVS-1 is superior to other strains due to its ability to utilize and compete for lactose-like carbohydrates.

Genomic and in vitro data have characterized the ability of the strain to consume lactose and lactose-like carbohydrates or produce beta-galactosidase (beta-gal or B-gal). Further, as indicated in the Examples provided herein, the Bifidobacterium adolescentis strain IVS-1 provides for the ability to metabolize lactose, reduce gas production in vitro, and reduce lactose intolerance symptoms.

In some aspects, the B. adolescentis strain IVS-1 provides superior B-gal activity to breakdown lactose. In embodiments, a colorimetric indicator of lactose degradation, such as, but not limited to, X-gal, ONPG, or similar, may be used in a liquid format to quantify enzyme activity through spectrophotometric measurement. The absorption may be standardized to CFU levels (or their proxy) and presented as relative levels normalized to other B-gal producing microbes. In aspects, the B. adolescentis strain IVS-1 may produce more B-gal activity to degrade lactose compared to other probiotic options and gut microbes.

In further aspects, B-gal gene expression may be measured through RT-qPCR with primers specific to the B-gal gene. Expression may be standardized to standard housekeeping gene levels and CFU enumeration (or their proxy) and presented as relative levels normalized to the lowest B-gal gene expressing microbe. In aspects, the B. adolescentis strain IVS-1 may express more B-gal activity to degrade lactose than other probiotic options and gut microbes.

As a further benefit, the B. adolescentis strain IVS-1 may deplete lactose from the environment faster than other probiotic options. In some aspects, lactose depletion rate can be measured by incubating a test microbe with a pre-set among of lactose and generating data standardized to CFU levels (or their proxy) and presenting it as relative levels normalized to other lactose degrading organisms. In aspects, the B. adolescentis strain IVS-1 may eliminate the lactose more quickly than other probiotic options and gut microbes. In other embodiments, B. adolescentis strain IVS-1 further outcompetes other organisms when performed with competitive lactose co-incubators in liquid cultures.

In embodiments, the compositions may be provided as a solid, liquid, or semi-solid composition. The solid compositions as described herein may be provided in the form of a tablet, a capsule, a powder, or a granulate (comprising a number of granules). In further embodiments, liquid and semi-solid compositions may be provided as a liquid suspension, a paste, or a syrup. A review of conventional formulation techniques can be found in e.g., Lachman et al. The Theory and Practice of Industrial Pharmacy. India: CBS Publishers & Distributors Pvt. Limited, 2009, which is herein incorporated by reference. In some aspects, the compositions may be prepared by methods known in the art and can be compressed, enterically coated, sugar coated, film coated or multiply compressed, containing suitable binders, lubricants, diluents, disintegrating agents, coloring agents, flouring agents, flow-inducing agents, and melting agents. Capsules, both soft and hard capsules, having liquid or solid contents, may be prepared according to conventional techniques that are well known in the pharmaceutical industry. As one example, the active probiotic organisms may be filled into gelatin capsules, using a suitable filling machine. In further embodiments, the solid composition as described herein may also be a pellet.

In embodiments, the compositions are preferably given to a subject by oral administration. In some aspects, oral delivery may be via low water activity (preferably below 0.2), not hot foods (preferably below 60° C.), and supplements. In embodiments, the food compositions can be any ingestible material, including but not limited to milk, curd, milk-based fermented products, acidified milk, yoghurt, desserts, snack foods, candy, lozenges, frozen yoghurt, milk powder, milk based powders, milk concentrate, cheese, cheese spreads, dressings, beverages, ice-creams, bars, additive powders, encapsulated and tableted supplements, fermented cereal based products, infant formulae, pet food, tablets, liquid bacterial suspensions, dried oral supplements, wet oral supplements, dry tube feeding or wet tube feeding that is produced by use of the Bifidobacterium adolescentis strain IVS-1, or derivative or a mutant strain thereof. In some embodiments, an acceptable carrier may be used to standardize the strain concentrations for appropriate dosing. For example, in one embodiment, the composition may be a chocolate truffle which may contain lactose (milk chocolate), or other dairy-containing ingredients.

The compositions or dosage forms may comprise at least Bifidobacterium adolescentis strain IVS-1 or a mutant strain thereof, as described above, so that the amount of the strain that is available for the individual is of about 10³ to about 10¹⁴ CFU per day, such as from about 10⁶ to about 10¹³ CFU per day, including from about 10⁸ to about 10¹² CFU per day, or even from about 10⁹ to about 10¹³ CFU per day. This amount may depend on the individual weight of the subject, and it is preferably of about 10⁹ to about 10¹² CFU/day for humans and about 10⁷ to about 10¹² CFU/day for pets. It will be understood, however, that the specific dose level for any particular subject will depend upon a variety of factors including the activity of the specific compound employed, the community structure and content of the microbiota, the age, body weight, general health, sex, diet, time of administration, route of administration, rate of excretion, formula combination, and the severity of the particular target condition of the subject.

In further embodiments, the compositions further comprise one or more prebiotic substances. Examples of suitable prebiotic substances include, but are not limited to, lactose, galactooligosaccharides (GOS), inulin, and other fructans. However other prebiotic substances such as fructooligosaccharides (FOS), mannan-oligosaccharides (MOS), arabinoxylans, and even a composition obtained from the bacterial cells, metabolites, enzyme activities, and polysaccharides of Bifidobacterium adolescentis strain IVS-1 are also contemplated.

Microorganisms are involved in the manufacture of food and feed products including most dairy products. Bacterial cultures, in particular cultures of bacteria generally classified as lactic acid bacteria, are essential in the making of all fermented milk products, cheese and butter. Cultures of these microorganisms are often referred to as starter cultures and impart specific features to various dairy products by performing a number of functions. In order for the starter culture to exert its function it is essential that it comprises live cells in sufficient amounts. Thus, some embodiments of the present disclosure provide a starter culture composition comprising living Bifidobacterium adolescentis strain IVS-1 or a mutant strain thereof, and wherein the starter culture composition preferably has a concentration of viable cells, which is in the range of from about 10⁴ to about 10¹² CFU per gram of the composition.

Starter cultures are typically used for the manufacturing of a food or feed product by adding the starter culture composition according to a food or feed product starting material and keeping the thus inoculated starting material under conditions where the lactic acid bacterium is metabolically active. In preferred embodiments, the food product is a milk-based product such as cheese, yoghurt, butter or a liquid fermented milk product, such as e.g., buttermilk or drinking yoghurt. The use of the present disclosure for the manufacturing of products containing cow milk or other dairy products is especially preferred.

Elimination and/or Reduction of Lactose Intolerance Symptoms

The present disclosure further provides methods of decreasing the symptoms of lactose intolerance during treatment with the IVS-1 strain and/or its mutant derivatives and also for an extended period of time after treatment stops. Thus, the methods of the present disclosure include partially, substantially, or completely decreasing the symptoms of lactose intolerance for a period of days, weeks, months, years, or permanently. Such a decrease is accomplished by the methods and compositions described herein.

Individuals who may benefit from the methods and compositions of the disclosure include individuals suffering from the symptoms of lactose intolerance, as described above. Any degree of lactose intolerance may be treated by the methods of the disclosure. Symptoms of lactose intolerance include gas, bloating, abdominal discomfort, diarrhea, vomiting, and/or cramping. Effectiveness of treatment may be measured in a number of ways. Conventional measurements, such as hydrogen gas production, stool acidity, symptom surveys, dietary recall surveys, microbiome measures, and/or blood glucose levels, may be used before and after treatment. Alternatively, or in addition, the amount of lactose that may be consumed before the onset of one or more symptoms may be measured or evaluated before and after treatment, thus, for example, treatment is considered at least partially effective if, after treatment, on average less hydrogen is produced with a given dose of lactose.

Alternatively, individuals will not precisely test the amount of hydrogen or, e.g., use a blood glucose test to measure effectiveness. Instead, individuals generally have a sense of how much lactose they may consume, and the types and degree of symptoms experienced after such consumption.

“Partial” elimination of symptoms of lactose intolerance is a statistically significant, increase in the amount of lactose that may be consumed before the onset of symptoms, a statistically significant decrease in the amount of breath hydrogen produces, or a statistically significant (P<0.05) reduction in reporting of some symptoms in digestive surveys. “Substantial” elimination of symptoms of lactose intolerance, as used herein, encompasses the above effects where the result is both statistically and clinically significant, as measured by appropriate effect size statistical evaluation. “Complete” or “substantially complete” elimination of symptoms of lactose intolerance, as used herein, indicates that normal amounts of lactose maybe consumed after treatment (i.e., the amount of lactose in a typical diet for the area and/or culture in which the individual normally lives) without symptoms, or with only the rare occurrence of symptoms. Thus, for example, an individual may know that if he or she consumes one half cup (4 oz.) of milk that there will be no, or minimal, symptoms, but if 1 or more cup of milk is consumed, then symptoms such as gas or diarrhea occur. The individual may find that, after treatment, 1 and one-half cups of milk may be consumed but that 3 or more cups cause symptoms, this indicates that symptoms of lactose intolerance were substantially eliminated. Alternatively, the individual may find that after treatment a normal diet for their geographical or cultural region may be consumed with no, or rare, symptoms of lactose intolerance. In that case, symptoms of lactose intolerance were completely eliminated.

More specifically, effectiveness may be measured by percent decrease in symptoms of lactose intolerance. In this measurement, the severity of a predetermined symptom, or set of symptoms is measured before and after treatment, e.g., using pre and post Likert scale. Exemplary symptoms include gas, bloating, diarrhea, cramping, abdominal pain, and vomiting. Any one, or more than one, of the symptoms may be measured. For example, an individual may be asked to rate one or more symptoms on a scale of increasing severity from 1 to 5. In one embodiment, a set of symptoms is rated, and the ratings are added; for example, gas, bloating, diarrhea, abdominal pain, and cramping may be rated. Percentage decrease in symptoms from before to after treatment may be calculated, and the symptoms of lactose intolerance may be considered eliminated by that percent decrease (e.g., if there is a 50% decrease in symptoms, then symptoms of lactose intolerance is 50% eliminated).

In some aspects, B. adolescentis strain IVS-1 may persist in subjects after about a 4-week washout. This is important in addressing lactose intolerance as the IVS-1 treatment can be provided to subjects with extra metabolic capacity in the gut microbiome to metabolize the lactose for a period of time, even if not taken every day. This is distinct from conventional lactase treatments, such as Lactaid® in which the treatment must be taken immediately prior to or with a lactose-containing meal. The IVS-1 strain of the present disclosure can serve to pre-condition the microbiome by increasing its persistence time within the gut of the subject. In some aspects, the B. adolescentis strain IVS-1 is further distinct from other probiotic strains that by and large pass through the gut quickly and do not colonize.

In some aspects, quantification of absolute cell numbers of IVS-1 in fecal samples may be conducted by qPCR using strain-specific primers for B. adolescentis IVS-1. In such aspects, IVS-1 has been shown to persist in several subjects, both with lactose added as well as with prebiotic GOS. In further aspects, 16S rRNA gene bacterial community sequencing of clinical trial participants has shown that an OTU matching B. adolescentis (OTU_1) increased during treatment with IVS-1 and remained higher than baseline after a 4-week washout. In aspects, for subjects receiving lactose added or additional prebiotic GOS, the B. adolescentis OTU_1 mean percent abundance was statistically significantly higher for the washout period than for the baseline.

In some embodiments, the present disclosure provides a method of decreasing symptoms of lactose intolerance in an individual exhibiting symptoms of lactose intolerance by administering to the individual increasing amounts of lactose for a period of time, wherein one or more symptoms of lactose intolerance are partially, substantially, or completely eliminated. In some embodiments, the symptom(s) of lactose intolerance remains partially, substantially, or completely eliminated for at least about 1 day, 1 week, 1 month, 2 months, 3 months, 4 months, 5 months, 6 months, 9 months, one year, 18 months, two years, three years, four years, five years, or more than five years after the termination of treatment, or permanently after the termination of treatment. In some embodiments, the present disclosure provides a method of decreasing symptoms of lactose intolerance in an individual exhibiting symptoms of lactose intolerance by administering to the individual increasing amounts of lactose for a period of time, wherein symptoms of lactose intolerance are substantially eliminated for at least about one month after treatment is terminated.

In some embodiments, the disclosure provides a method of decreasing symptoms of lactose intolerance in an individual exhibiting symptoms of lactose intolerance by administering to the individual increasing amounts of lactose for a period of time, wherein the symptoms of lactose intolerance, measured as described herein, are decreased by an average of about 10%, 20%, 30%, 40%, 50%, 60%, 70%, 80%, 90%, 95%, or about 100%. An “average” decrease is a decrease as measured in a group of individuals exhibiting symptoms of lactose intolerance, such as more than about 2, 3, 4, 5, 10, 25, or 50 individuals. In some embodiments, the decrease of symptoms of lactose intolerance persists or becomes even greater (e.g., 50% decrease to 55% decrease) for at least about 1 day, 1 week, 1 month, 2 months, 3 months, 4 months, 5 months, 6 months, 9 months, one year, 18 months, two years, three years, four years, five years, or more than five years after the termination of treatment. In some embodiments, the decrease in symptoms is permanent. In some embodiments, the present disclosure provides a method of decreasing symptoms of lactose intolerance in an individual exhibiting symptoms of lactose intolerance by administering to the individual increasing amounts of lactose for a period of time, wherein the symptoms of lactose intolerance, measured as described herein, are decreased by an average of about least about 20% and remain decreased by at least about 20% for at least about one month after treatment is terminated. In some embodiments, the present disclosure provides a method of decreasing symptoms of lactose intolerance in an individual exhibiting symptoms of lactose intolerance by administering to the individual increasing amounts of lactose for a period of time, wherein the symptoms of lactose intolerance, measured as described herein, are decreased by an average of at least about 50% and remain decreased by at least about 50% for at least about one month after treatment is terminated.

The total duration of treatment may be from about 2 weeks to about 4 weeks, or about 1 week to about 10 weeks, or about 4 weeks to about 8 weeks, or about 3 weeks. During this period of time, the subject is started on a program of treatment, and subsequently challenged with a lactose-containing product after the intervention. In some embodiments, treatment with the IVS-1 strain is combined with one or more additional substances such as prebiotics, as described herein. In some embodiments, the total duration of treatment is about 1 day to about 30 days, or about 5 days to about 10 days, or about 2 days to about 15 days, or about 1 day to about 4 days, or about 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, the total duration of treatment is about 3 days. In some embodiments, the total duration of treatment is about 14 days. It will be appreciated that these durations are averages, and that individuals using the treatment may vary from the average based on the severity of their symptoms, their existing microbiome content, missing days of treatment, and the like. In some embodiments, the duration of the treatment is based on the individual's symptoms or existing microbiome content. Thus, an individual may experience a return of symptoms at a given intake of lactose, and may require an adjustment of treatment dose and/or time, or a lowering of their intake of lactose, until symptoms subside. Thus, in some embodiments, the duration of the treatment is not definitively established at the outset, but continues until the highest dose of lactose is achieved, or until the desired level of lactose tolerance is achieved.

Increasing dosage of Bifidobacterium adolescentis strain IVS-1 may be achieved by increasing the amount of Bifidobacterium adolescentis strain IVS-1 administered, either by increasing the amount of Bifidobacterium adolescentis strain IVS-1 per dose, the number of doses, or both. In one embodiment, both strategies are used. Thus, in some embodiments of the disclosure, Bifidobacterium adolescentis strain IVS-1 is initially administered with every meal, followed by reduction to once per day administration. The once per meal administration can last for a period of about 1 to about 30, about 1 to about 10, about 2 to about 20, about 6 to about 20, or about 20 days, and the once per day administration can last for a period of about 1 to about 8, about 2 to about 10, about 2 to about 12, about 4 to about 14, about 16 days, or indefinitely if required to maintain remission of symptoms. In some embodiments, during the once per meal administration period, the intake of lactose is minimized. In some embodiments, lactose intake may be ramped up after one, three, five, or 7 days, or after 2, 3, or 4 weeks, or any combination thereof.

After treatment is concluded, an individual subject is encouraged to enjoy dairy products or other lactose-containing foods regularly, such as every other day, twice a week, or once a week in order to maintain colonization by the IVS-1 strain, and thus, the reduction in symptoms of lactose intolerance.

While an individual typically will not require more than one course of treatment, in some embodiments, an individual may have repeated courses of treatment. The course of treatment may be repeated when symptoms of lactose intolerance appear or increase to an undesirable level. In one embodiment, a “booster pack” of the IVS treatment may be recommended in order to recolonize individuals who have allowed their lactose consumption to lapse and thus are susceptible to experiencing symptoms again. Alternatively, the course of treatment may be repeated at regular or predetermined intervals. Thus, treatment may be repeated after about one month, two months, three months, four months, six months, eight months, ten months, one year, 18 months, two years, three years, four years, five years, or more than five years, or any combination thereof (e.g., treatment may be repeated after one year, then every two to five years thereafter). The treatment may be repeated in the same form (e.g., duration, dosage, timing of dosage, additional substances, etc.) as used in the first treatment, or it may be modified. For example, treatment duration may be shortened or lengthened, dosage may be increased more quickly or slowly and/or a higher or lower starting dose of lactose may be used, a different lactose-containing product may be used (e.g., containing more or less of other substances, or fewer or more substances in addition to lactose), and the like. In another embodiment a “maintenance” dosage may be offered comprising a lower dosage than normal that is administered daily to prevent symptoms from occurring.

In Vivo Selection (IVS)

The present disclosure further provides for methods of selecting and isolating one or more microbial strains that are naturally occurring in the gastrointestinal microbiota and that exhibit improved characteristics in the presence of lactose. Following isolation and characterization, the in vivo-selected microbial strain can be used as a therapeutic and, optionally along with the prebiotic, can be administered as a synbiotic composition to an animal. For purposes herein, gastrointestinal microbiota refers to the microbial population that is present in the gastrointestinal tract of a subject. The gastrointestinal tract typically includes the mouth, esophagus, stomach, small intestine, large intestine, rectum and anus.

In one embodiment, methods are provided in which a microbial strain that is present in the gastrointestinal microbiota of one or more subjects can be specifically selected based on its positive response to the presence of a prebiotic. Such methods typically start with the administration of a candidate prebiotic to a subject.

Typically, a subject is administered at least one dose of the prebiotic (e.g., lactose or GOS) but, more often, a number of doses over a period of time (e.g., one or more doses per day for multiple days (e.g., for about or at least a week, for about or at least two weeks)). In some instances, to help or further validate the correlation between an increase in a microbial strain and the presence of lactose, the subject can be administered a prebiotic in sequentially higher doses over time.

Using the methods described herein, additional microbial strains can be identified, based on in vivo selection, which preferentially increases in number, or otherwise causes a positive response in, specific strains due to the presence of a prebiotic such as lactose or GOS. In addition to an increase in number of one or more microbial strains in the presence of a prebiotic relative to a baseline sample, a “positive response” can refer to, for example, an increase in metabolic activity by the microbial strain (i.e., in the absence of an increase in number) or both an increase in number and an increase in metabolism of lactose.

As described herein, this method has been used to identify a microbial strain that responds particularly well to a prebiotic (i.e., lactose or GOS). As described herein, this microbial strain was identified as a Bifidobacterium adolescentis strain IVS-1. This strain was deposited with the American Type Culture Collection (ATCC, 10801 University Blvd., Manassas, Va. 20110) on Oct. 8, 2013, and assigned Accession No. PTA-120614.

The Bifidobacterium adolescentis strain IVS-1 or other strains identified using the methods herein can be provided as a substantially pure population. As used herein, a “substantially pure population” of cells means that at least about 50% (e.g., about 55%, 60%, 65%, 70%, 75%, 80%, 90%, 99% or greater) of the cells present are the Bifidobacterium adolescentis strain IVS-1 described herein. Methods of culturing Bifidobacterium adolescentis strain IVS-1 would be known to those of skill in the art. See, for example, Handbook of Culture Media for Food Microbiology, 2nd Ed., Vol 37, Corry et al., eds., 2003, Elsevier Science. In addition, there is a commercially available selective medium defined specifically for culturing Bifidobacterium (e.g., MRS media from Difco).

After selecting, identifying and isolating a microbial strain using the methods disclosed herein, and after confirming the microbial strains affinity for the prebiotic (e.g., GOS or lactose), the microbial strain can be administered to a subject (e.g., as a probiotic). Given the method by which the microbial strain was obtained, it is preferred that the microbial strain be administered to the subject in conjunction with the corresponding prebiotic (e.g. GOS lactose). In some embodiments, the microbial strain and the prebiotic are combined prior to administration to produce a synbiotic. Typically, the animal that is administered the rationally-designed synbiotic is of the same species as the subject from which the microbial strain originally was identified. As described above with respect to the subjects, the subject may be an animal, including humans or any number of non-human animals.

In some embodiments, the microbial strain and the prebiotic (e.g., the synbiotic) can be contained within a foodstuff. Foodstuffs include any number of food products that are suitable for human consumption such as, without limitation, milk, yogurt, juices, water, cereals, chewing gum, crackers, candies, cookies, vitamin supplements, meats, and fruits or vegetables (i.e., blended fruits or vegetables such as, e.g., baby food). Foodstuffs also include feed products (e.g., suitable for consumption by livestock or companion animals) including dry animal feeds. In certain embodiments, the Bifidobacterium adolescentis strain IVS-1 described herein along with GOS can be mixed into liquid feed or drinking water, or combined with a carrier and applied to solid feed.

A composition or a foodstuff that includes the microbial strain (e.g., Bifidobacterium adolescentis strain IVS-1) and the prebiotic (e.g., lactose or GOS) as described herein can further include a pharmaceutically acceptable carrier. A pharmaceutically acceptable carrier should be non-toxic to the bacteria and to the animal, and also can include an ingredient that promotes viability of the microorganism during storage. Liquid or gel-based carriers are well known in the art, such as water, fruit juice, glucose or fructose solutions, physiological electrolyte solutions, and glycols such as methanol, ethanol, propanol, butanol, ethylene glycol and propylene glycol. Carriers also include oleaginous carries such as, for example, white petrolatum, isopropyl myristate, lanolin or lanolin alcohols, mineral oil, fragrant or essential oil, nasturtium extract oil, sorbitan mono-oleate, cetylstearyl alcohol, hydroxypropyl cellulose (MW=100,000 to 1,000,000), or detergents (e.g., polyoxyl stearate or sodium lauryl sulfate). Other suitable carriers include water-in-oil or oil-in-water emulsions and mixtures of emulsifiers and emollients are provided.

A composition or a foodstuff that includes the microbial strain (e.g., Bifidobacterium adolescentis strain IVS-1) and the prebiotic (e.g., lactose or GOS) as described herein also can include natural or synthetic flavorings and food-quality coloring agents, thickening agents such as corn starch, guar gum, xanthan gum and the like, binders, disintegrators, coating agents, lubricants, stabilizers, solubilizing agents, suspending agents, excipients, and diluents. Additional components also can be included that, for example, improve palatability, improve shelf-life, and impart nutritional benefits. It would be understood by those skilled in the art that any additional components in a composition must be compatible with maintaining the viability of the microbial strain.

Administration of a composition or a foodstuff that includes the microbial strain (e.g., Bifidobacterium adolescentis strain IVS-1) and the prebiotic (e.g., lactose or GOS) as described herein can be accomplished by any method that delivers at least a portion of the microorganisms and prebiotic into the digestive tract of an animal. Therefore, enteral administration is preferred (e.g., orally, sublingually, or rectally), although other routes are not excluded. Generally, the formulation of a composition is dependent upon its intended route of delivery. For example, a composition that includes the Bifidobacterium adolescentis strain IVS-1 (with or without lactose or GOS) as described herein can be formulated as a powder, a granule, a tablet, a capsule, a liquid suspension, a paste, or a syrup.

Probiotics are reported to produce health benefits which include (1) alleviation of intestinal disorders such as constipation and diarrhea caused by infection by pathogenic organisms, antibiotics, or chemotherapy; (2) stimulation and modulation of the immune system; (3) anti-tumor effects due to inactivation or inhibition of carcinogenic compounds in the gastrointestinal tract by reduction of intestinal bacterial enzyme activities such as beta-glucuronidase, azoreductase, and nitroreductase; (4) reduced production of toxic end products such as ammonia, phenols and other metabolites of protein known to influence liver cirrhosis (5) reduction in serum cholesterol and blood pressure; (6) maintenance of mucosal integrity; (7) alleviation of symptoms of lactose intolerance; and (8) prevention of vaginitis. Accordingly, the beneficial effects attributed to probiotics include increased resistance to infectious diseases, healthier immune systems, reduction in irritable bowel syndrome, reductions in blood pressure, reduced serum cholesterol, milder allergies and tumor regression. In animals, for example, probiotics can enhance weight gain or weight loss and improve meat quality, and milk production. Significantly, probiotics can be used to establish and maintain a healthy (e.g., balanced) gastrointestinal flora in an animal and to reduce the effect of gastrointestinal diseases. Gastrointestinal diseases include, without limitation, diarrhea, constipation, loose stool, abdominal inflation, ulcerous colitis, Crohn's disease, irritable bowel syndrome, hypersensitive intestinal syndromes, food toxicity, food allergy, pseudomembranous colitis, hemorrhagic colitis, gastritis, gastroduodenal ulcer, dental caries, and periodontitis. In aspects, bifidobacteria may prevent or reduce the effects of metabolic disorders such as obesity and type 2 diabetes by reducing gut permeability. Reducing gut permeability can improve metabolic endotoxemia and metabolic inflammation, both of which are involved in obesity and related metabolic disorders.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. However, those of skill in the art should, in light of the present disclosure, appreciate that many changes can be made in the specific embodiments which are disclosed and still obtain a like or similar result without departing from the spirit and scope of the disclosure.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Genomic Characterization

The genome of the Bifidobacterium adolescentis IVS-1 strain was sequenced via 454 Pyrosequencing to determine whether or not the strain contained a B-galactosidase gene for lactose metabolism. The genome was submitted to NCBI and analyzed by the NCBI prokaryotic genome annotation automated computational analysis pipeline. Based on the results, the IVS-1 strain was confirmed to contain a B-galactosidase gene for lactose metabolism (see https://www.ncbi.nlm.nih.gov/protein/KIM01121.1?report=genpept).

### Example 2

### In Vivo Characterization

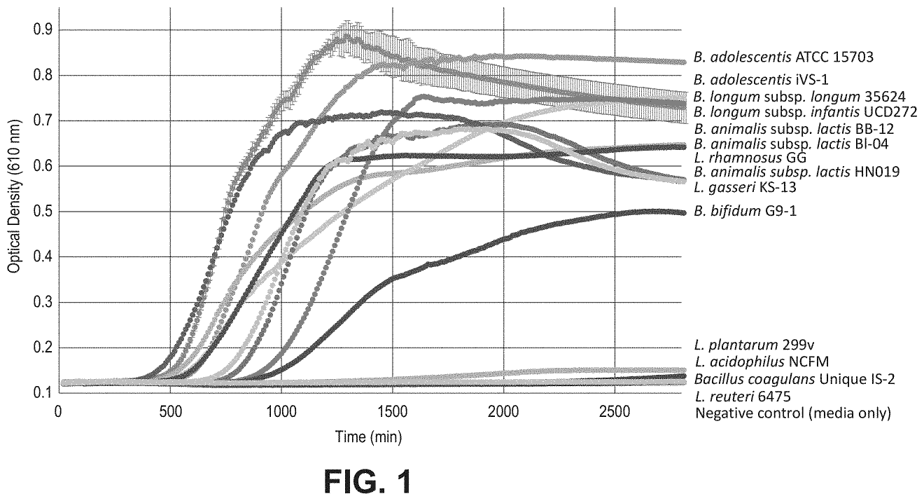

The growth of Bifidobacterium adolescentis strain IVS-1 was evaluated in comparison to a number of other probiotic strains in a simulated gastrointestinal environment containing lactose. The optical density (OD 600) of each strain was measured during growth on 1% lactose with an inoculum of approximately 10⁴ CFU into gut-like media. Growth was completed in wells of a 96-well plate incubated in a plate reader in an anaerobic chamber. The data showing the growth of each of the probiotic strains is provided in FIG. 1.

The data provided in FIG. 1 provides the average of four replicates for each strain. For clarity, the standard deviation is shown only for B. adolescentis IVS-1 strain, however, the replicate variation was similar for other strains shown in FIG. 1. The probiotic strains were prepared ahead of time by growing in liquid MRS or RCM (both rich medias) to late log phase, centrifuging, resuspending pellets in gut-like media containing 7.5% DMSO as a cryopreservative. The aliquots were then frozen.

As shown within FIG. 1, the IVS-1 strain was confirmed to grow much faster than other strains in the simulated gastrointestinal environment containing lactose. Without being limited to a particular mechanism or theory, the increased growth rate of the IVS-1 strain may contribute to its superior properties in reducing gas.

### Example 3

### Gas Production in Fecal Fermentations

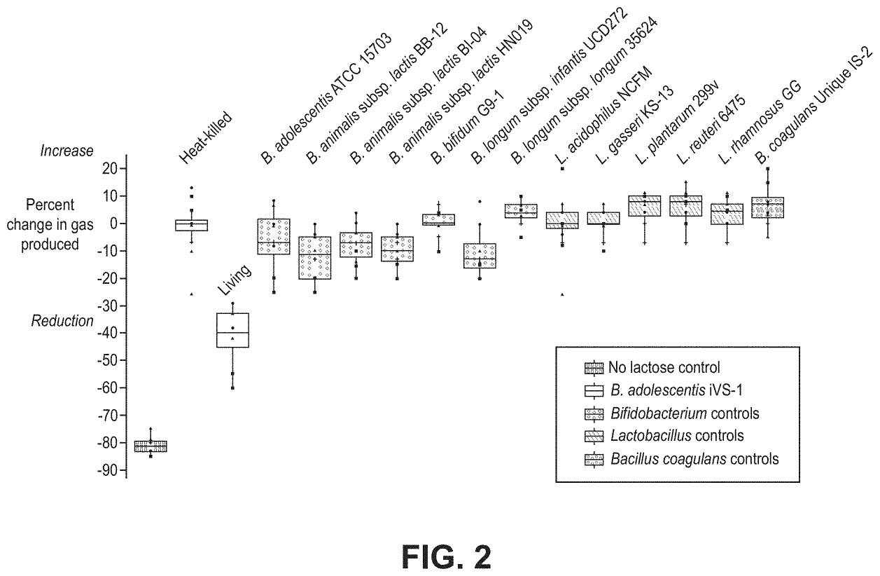

The amount of gas produced and the relative change in gas production were evaluated with B. adolescentis strain IVS-1 and a number of additional probiotics. The measurement of gas production was following inoculation of a gut-like media containing 1% lactose in diluted fecal samples. About 10⁷ CFU of a range of different probiotics were utilized in the analysis. The change in gas production was compared to controls containing no probiotics as shown in FIG. 2. Probiotic strains were prepared ahead of time by growing them in liquid MRS or RCM (both rich medias) to late log phase, centrifuging them, resuspending pellets in gut-like media containing 7.5% DMSO as a cryopreservative, and then freezing aliquots of each strain. The heat-killed IVS-1 control was incubated at 70° C. for 5 minutes prior to being added. The experiments were conducted in triplicate, with each of four different fecal donors. The individual measurements for each of the 4 fecal donors are denoted as circles, triangles, squares, or crosses within FIG. 2.

As shown in FIG. 2, B. adolescentis IVS-1 reduced gas from lactose significantly more than other strains. The results demonstrated that the IVS-1 strain provided superior results in this pre-clinical model of gas produced from gastrointestinal lactose metabolism. As excess gas production is a main cause of lactose intolerance symptoms, the results indicate that B. adolescentis strain IVS-1 may be used in treating lactose intolerance by reducing the amount of gas produced.

### Example 4

### Hydrogen Production and Symptom Score Reduction

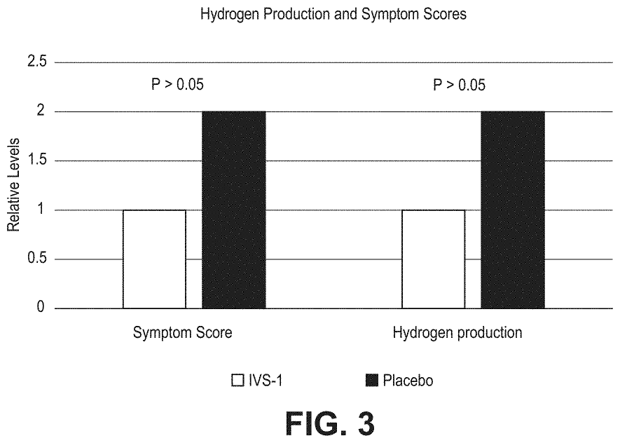

A clinical trial on B. adolescentis strain IVS-treatment is being conducted to analyze the hydrogen product and symptoms score reduction on individuals receiving lactose. It is expected that the IVS-1 treatment will be superior to placebo in terms of the resultant gas levels and lactose intolerance symptom scores, as shown in FIG. 3. Breath hydrogen will be measured using a handheld breath meter, and symptom scores will be based on subjective severity and/or frequency surveys evaluated by the study subject themselves.

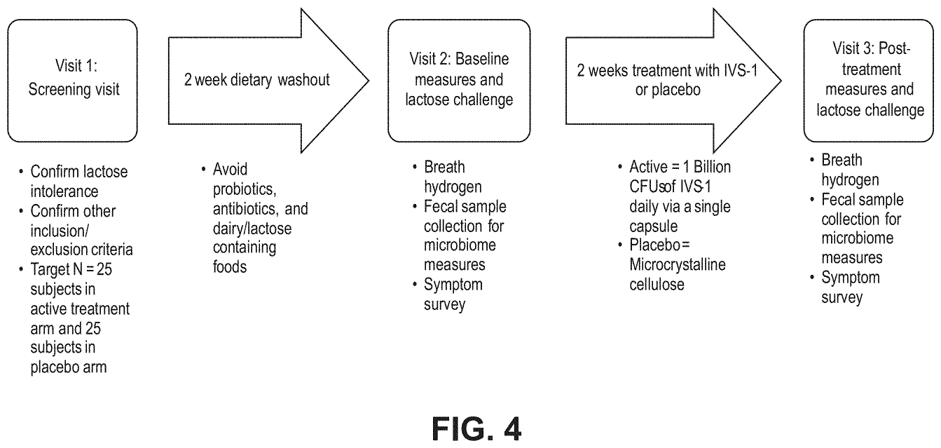

The hydrogen breath test: This will be an 8-hour hydrogen breath test. Pre-qualified participants shown to be lactose intolerant will fast for 12 hours prior to the test and then be given commercial milk to drink. Each subject will be fed milk containing 0.5 g lactose per kg body weight assuming a lactose content of 5% (12 g per 240 ml cup) with a maximum of 50 g of lactose. This dose of lactose is in the physiological range of typical dietary consumption and has historically resulted in some elevation of symptoms. Breath samples measuring hydrogen levels will be taken at the following time points: 0 (pre-milk dose), 30 minutes, 60 minutes, 2 hours, 3 hours, 4 hours, 5 hours, 6 hours, 7 hours, and 8 hours for a total of ten breath samples. Participants will be asked to record any symptoms they experience related to lactose intolerance at these same time intervals using provided forms. A further flowchart of the procedure can be found in FIG. 4.

### Example 5

### Relative Lactase Activity of Various Probiotics/Growth Conditions

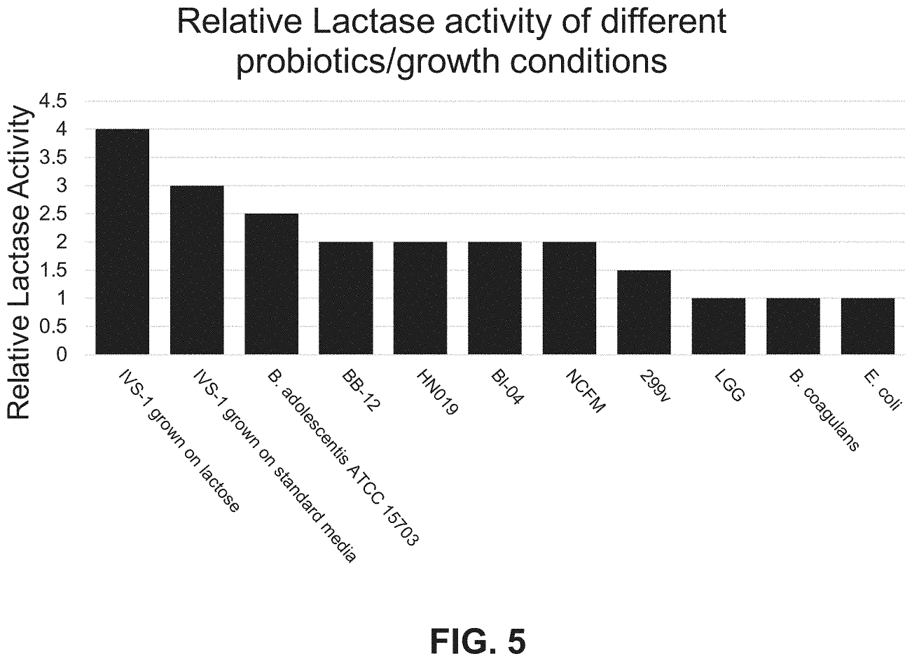

In this study, a colorimetric indicator of lactose degradation (X-gal, ONPG, or similar) will be used in a liquid format and mixed in a standardized amount with various strains grown in broth media to quantify lactase (B-gal) enzyme activity through spectrophotometric measurement of color change. The various strains will be as shown in FIG. 5. Absorption will be standardized to CFU levels (or their proxy) and presented as relative levels normalized to the lowest lactase activity producing microbe in the study. The anticipated results shown in FIG. 5 demonstrates that IVS-1 will likely produce more B-gal activity to degrade lactose than other probiotic options. Further, the amount of lactase activity is expected to be higher when IVS-1 is provided with lactose during growth in preparation of the strain for the study. The pre-treatment of IVS-1 with lactose will provide synergistic effects with regard to the growth of IVS-1 and lactase activity.

All publications and patent applications mentioned in this specification are herein incorporated by reference to the same extent as if each individual publication or patent application was specifically and individually indicated to be incorporated by reference.

While the disclosure has been described in connection with various embodiments, it will be understood that the disclosure is capable of further modifications. This application is intended to cover any variations, uses or adaptations of the disclosure following, in general, the principles of the disclosure, and including such departures from the present disclosure as, within the known and customary practice within the art to which the disclosure pertains.

What is claimed is:

1. A method of treating lactose intolerance, comprising: administering to a subject in need thereof a therapeutically effective amount of an isolated Bifidobacterium adolescentis strain IVS-1.

2. The method of claim 1, wherein the strain is administered in an amount of at least 10⁶ CFU per day.

3. The method of claim 2, wherein the strain is administered daily for one week or longer.

4. The method of claim 3, wherein the method improves lactose degradation and/or decreases lactose intolerance symptoms in a subject in need thereof.

5. The method of claim 1, wherein the Bifidobacterium adolescentis strain IVS-1 is pre-treated with lactose to increase initial lactase activity of the Bifidobacterium adolescentis strain IVS-1.

6. The method of claim 1, wherein the method provides lactase to a subject in need thereof via administration of both live and inactive cultures of Bifidobacterium adolescentis strain IVS-1 and the actual and former cellular contents thereof. 7 The method of claim 1, wherein the method reduces gas production in the intestines.

8. The method of claim 1, wherein the method balances osmotic pressures in the intestines.

9. The method of claim 1, wherein the method alters the intestinal microbiome to benefit the health and wellbeing of a subject in need thereof.

10. A food composition to treat lactose intolerance, comprising Bifidobacterium adolescentis strain IVS-1, wherein the inoculum of Bifidobacterium adolescentis strain IVS-1 demonstrates lactase activity.

11. The food composition of claim 10, wherein the food composition is selected from the group consisting of milk, curd, milk based fermented products, acidified milk, yoghurt, desserts, snack foods, chocolates, candy, lozenges, frozen yoghurt, milk powder, milk based powders, milk concentrate, cheese, cheese spreads, dressings, beverages, ice-creams, bars, additive powders, encapsulated and tabled supplements, fermented or non-fermented cereal based products, infant formulae, tablets, liquid bacterial suspensions, dried oral supplement, and wet oral supplement.

12. The composition of claim 10, further comprising a pharmaceutically acceptable carrier.

13. The composition of claim 10, further comprising a prebiotic selected from the group consisting of: galactooligosaccharides (GOS), fructooligosaccharides (FOS), and inulin, mannan-oligosaccharides (MOS), arabinoxylans, or a composition obtained from the bacterial cells, metabolites, enzyme activities, and polysaccharides of Bifidobacterium adolescentis strain IVS-1.

14. The composition of claim 10, wherein Bifidobacterium adolescentis strain IVS-1 is selected from a wild type strain, a derived strain, and/or a mutant strain.

15. The composition of claim 10, wherein Bifidobacterium adolescentis strain IVS-1 is at a concentration at about 10⁴ to about 10¹⁴ CFU.

16. A method of treating lactose intolerance, comprising: administering to a subject in need thereof a therapeutically effective amount of a Bifidobacterium adolescentis strain IVS-1; and increasing the lactase activity by Bifidobacterium adolescentis strain IVS-1 in the gastrointestinal tract after at least 1 day of administration.

17. The method of claim 16, further comprising increasing the beta-galactosidase activity levels in the gastrointestinal tract via administration of Bifidobacterium adolescentis strain IVS-1.

18. The method of claim 16, further comprising decreasing the lactose levels in the gastrointestinal tract via administration of Bifidobacterium adolescentis strain IVS-1.

19. The method of claim 16, further comprising delivering an effective amount of GOS with the Bifidobacterium adolescentis strain IVS-1.

20. The method of claim 16, further comprising decreasing hydrogen production in the gastrointestinal tract and reducing symptoms related to lactose intolerance in the subject.


SEQUENCE LISTING (1 sequence)

SEQ ID NO: 1 - DNA, 1288 bp, Bifidobacterium adolescentis
  source 1..1288: mol_type = genomic DNA
tgcagtcgaacgggatcccaggagcttgctcctgggtgagagtggcgaacgggtgagtaatgcgtgaccgacctgccccatacaccggaatagctcctggaaacgggtggtaatgccggatgctccagttgaccgcatggtcctctgggaaagcttttgcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgctcttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatacggtctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacactgtgaggtggagcggatcccttaaaaccggtctcagttcggattggagtctgcaacccgactccatgaaggcggagtcgctagtaatcgcggatcag